(19) United States
(12) Patent Application Publication
(10) Pub. No.: US 2014/0238395 A1
(43) Pub. Date: Aug. 28, 2014

(54) POWDER INHALING DEVICE

(71) Applicant: Arnaud Colomb, Verneuil sur Seine (FR)

(72) Inventor: Arnaud Colomb, Verneuil sur Seine (FR)

(73) Assignee: APTAR FRANCE SAS, Le Neubourg (FR)

(21) Appl. No.: 14/238,621

(22) PCT Filed: Aug. 13, 2012
(86) PCT No.: PCT/FR2012/051884

§ 371 Date: Feb. 12, 2014

(30) Foreign Application Priority Data
Aug. 19, 2011 (FR) 1157414

(51) Int. Cl. A61M 15/00

(52) U.S. Cl. CPC A61M 15/0005; A61M 15/0051; A61M 15/0086; A61M 15/0043; A61M 15/0055
USPC 128/203.15

(57) ABSTRACT

A powder inhaler device having a body provided with a dispenser orifice; a plurality of predosed reservoirs each containing a dose of powder for dispensing; and reservoir-opening device for opening a reservoir on each actuation. The inhaler device has a dispersion chamber including an inlet connected during inhalation to an open reservoir and receiving the flow of air and of powder from the open reservoir via a delivery channel, and an outlet connected to the dispenser orifice via a dispenser channel. The dispersion chamber including at least one ball that is movable along a ball path in the dispersion chamber, the dispersion chamber including at least one air inlet that is approximately tangential to said ball path, the delivery and dispenser channels extending in a same direction that is substantially perpendicular to the ball path and to the tangential air inlet.

The present invention relates to a powder inhaler device, and more particularly to a dry-powder inhaler.

Inhalers are well known in the prior art. Various types exist. A first type of inhaler contains a reservoir receiving many doses of powder, the inhaler being provided with metering means making it possible, on each actuation, to remove one dose of said powder from the reservoir, so as to bring said dose into an expulsion duct in order to be dispensed to the user. Inhalers including individual reservoirs, such as capsules, that are loaded into the inhaler just before said reservoir is used are also described in the prior art. The advantage of such devices is that it is not necessary to store all of the doses inside the appliance, such that said appliance can be compact. However, the inhaler is more difficult to use, since the user is obliged to load a capsule into the inhaler before each use. Another type of inhaler consists in packaging the doses of powder in individual predosed reservoirs of the blister type, then in opening one of the reservoirs each time the inhaler is actuated. That implementation seals the powder more effectively since each dose is opened only when it is about to be expelled. In order to make such individual reservoirs, various techniques have already been proposed, such as an elongate blister strip or blisters disposed on a rotary circular disk. All existing types of inhalers, including those described above, present both advantages and drawbacks associated with their structures and with their types of operation. Thus, with certain inhalers, there is the problem of metering accuracy and reproducibility on each actuation. In addition, the effectiveness of the dispensing, i.e. the fraction of the dose that effectively penetrates into the user's lungs in order to have a beneficial therapeutic effect, is also a problem that exists with a certain number of inhalers. A solution for solving that specific problem has been to synchronize the expulsion of the dose with the inhalation of the patient. However, this makes the devices complex and thus costly to manufacture and to assemble. Document WO 2010/004229 describes an inhaler including a dispersion chamber, the flow of air loaded with powder entering into said dispersion chamber via a tangential inlet and leaving said dispersion chamber via an outlet that is perpendicular to said tangential inlet.

An object of the present invention is to provide a fluid dispenser device, in particular a dry-powder inhaler, that does not have the above-mentioned drawbacks.

In particular, an object of the present invention is to provide such an inhaler that is simple and inexpensive to manufacture and to assemble that is reliable in use, with good metering accuracy that is reproduced on each actuation.

The present invention thus provides a powder inhaler device comprising: a body that is provided with a dispenser orifice; a plurality of predosed reservoirs each containing a dose of powder for dispensing; and reservoir-opening means for opening a reservoir on each actuation; the device further comprising a dispersion chamber including an inlet connected during inhalation to an open reservoir and receiving the flow of air and of powder from said open reservoir via a delivery channel, and an outlet connected to said dispenser orifice via a dispenser channel, said dispersion chamber including at least one ball that is movable along a ball path in said dispersion chamber, said dispersion chamber including at least one air inlet that is approximately tangential to said ball path, said device being characterized in that said delivery and dispenser channels extend in a same direction that is substantially perpendicular to said ball path and to said at least one tangential air inlet.

In a first advantageous variant, said delivery channel is substantially coaxial with said dispenser channel, said inlet and outlet of the dispersion chamber being arranged substantially at the center of said dispersion chamber.

Advantageously, said inlet of the dispersion chamber includes a deflector so as to deflect the flow of air and of powder towards the ball path of said dispersion chamber.

Advantageously, said deflector is dish shaped with the outer tip of said dish facing the incoming flow of air and of powder.

In a second advantageous variant, said delivery channel is offset relative to said dispenser channel, said inlet of the dispersion chamber being arranged at the ball path, and said outlet of the dispersion chamber being substantially at the center of said dispersion chamber.

Advantageously, said inlet of the dispersion chamber includes a mesh.

Advantageously, each air inlet of the dispersion chamber is formed by a tangential air channel.

Advantageously, said plurality of reservoirs is formed on an elongate flexible blister strip comprising a base layer containing the cavities of the reservoirs, and a closure layer overlying said cavities.

Advantageously, the closure layer is peelable off the base layer, the portion of base layer containing the empty blisters rolling up around a first rotary receiver element, and the portion of closure layer peeled off said base layer rolling up around a second rotary receiver element.

Advantageously, said reservoir opening means include means for unsticking said closure layer from said base layer, such as a peeling edge around which said closure layer extends, the device including displacement means, such as an indexer wheel, so as to cause the blister strip to advance before and/or during each actuation, the downstream end of said closure layer being fastened to a second rotary receiver element that rotates in correlation with said indexer wheel.

Advantageously, said dispersion chamber contains a plurality of balls.

Advantageously, after opening, said open reservoir is situated facing both said delivery channel and an air delivery channel, such that during inhalation, a flow of air penetrates into said blister through said air delivery channel and entrains said powder in a flow of air and of powder via said delivery channel towards said dispersion chamber.

These characteristics and advantages and others of the present invention appear more clearly from the following detailed description, given by way of non-limiting example, and with reference to the accompanying drawing, and in which:

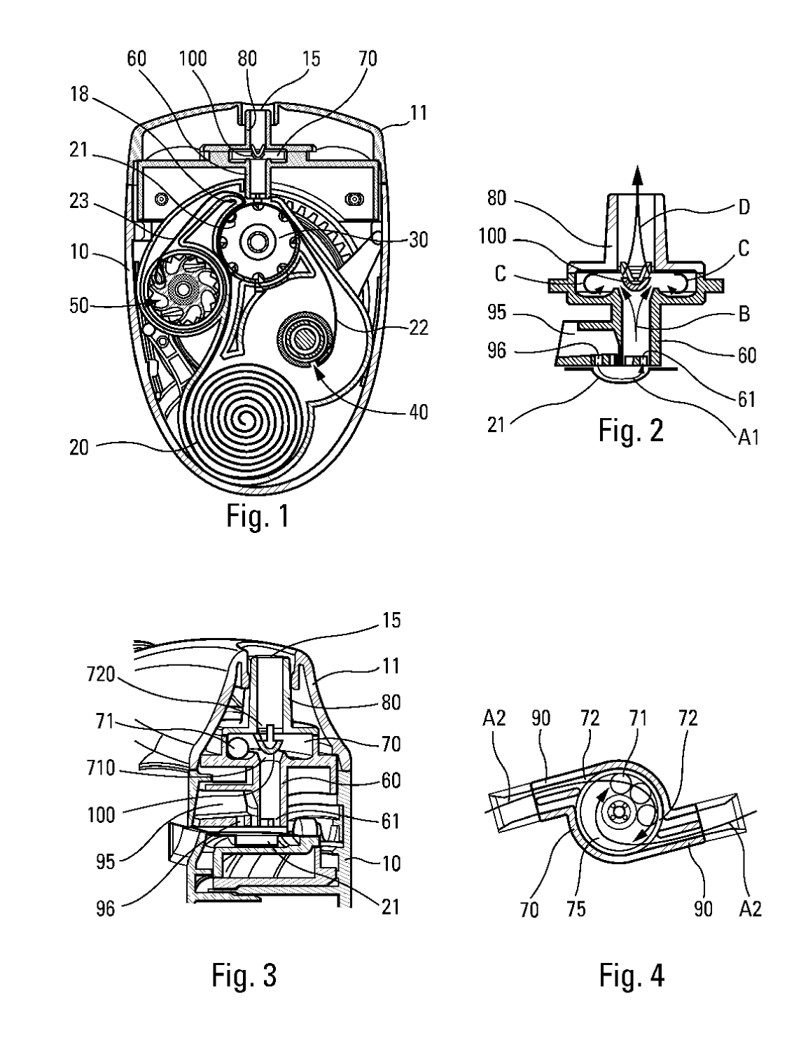

FIG. 1 is a diagrammatic section view of a powder inhaler device in a first advantageous embodiment of the invention;

FIG. 2 is a cross-section view of a detail of a portion of the FIG. 1 device;

FIG. 3 shows a cut-away diagrammatic perspective view of the portion of the inhaler device shown in FIG. 2;

FIG. 4 is a horizontal diagrammatic section view of the dispersion chamber.

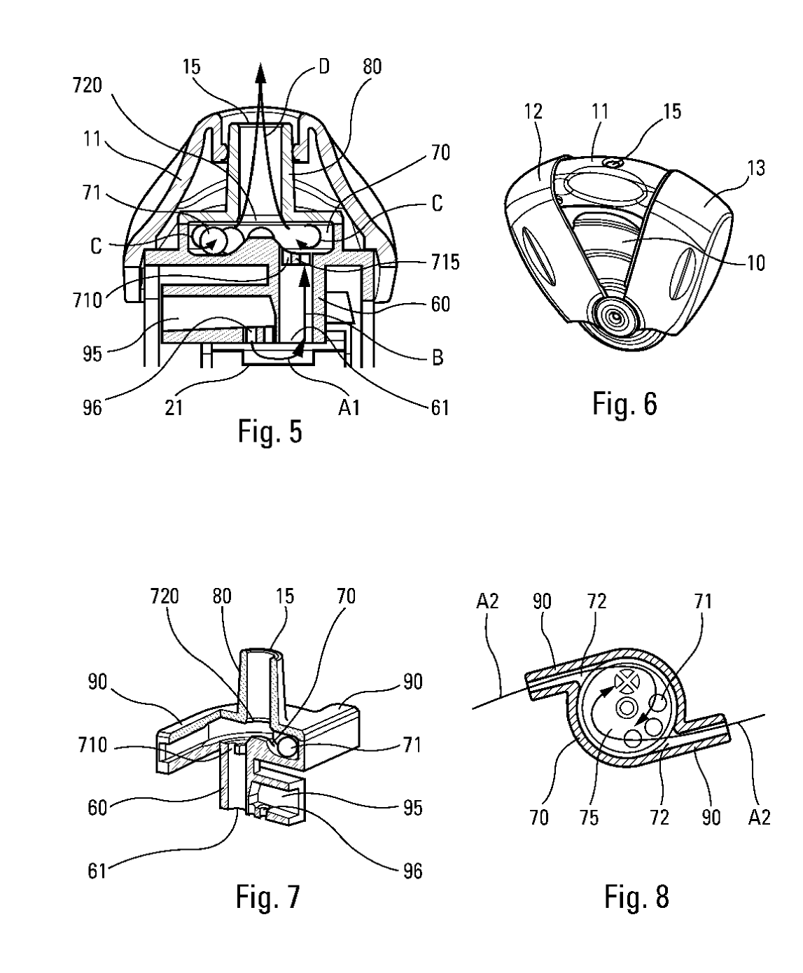

FIG. 5 is a view similar to the view in FIG. 2 showing in part a second advantageous embodiment of the invention;

FIG. 6 is a perspective view of the device with the cover portions open;

FIG. 7 is a view similar to the view in FIG. 3, showing the second embodiment; and

FIG. 8 is a view similar to the view in FIG. 4.

FIG. 1 shows an advantageous variant embodiment of a dry-powder inhaler. The inhaler includes a body 10 on which there can be slidably or pivotally mounted two cover-forming portions 12, 13 that are visible in FIG. 6 and that are adapted to be opened so as to open and prime the device. In a variant, a single cover portion is also possible. The body 10 can be approximately rounded in shape, but it could be of any other appropriate shape. The body 10 includes a mouthpiece 11 that defines a dispenser orifice 15 through which the user inhales while the device is being actuated.

Inside the body 10 there is provided a strip 20 of individual predosed reservoirs 21, also known as blisters, said strip being made in the form of an elongate flexible strip on which the blisters are arranged one behind another, in manner known per se. The blister strip 20 is of the peelable type with a base layer 22 including the cavities forming the blisters, and a peelable closure layer 23 that progressively unsticks from the base layer 22 on each actuation.

Before first use, the blister strip 20 can be rolled up inside the body 10, preferably in a storage portion, and displacement means 30 for displacing the strip are provided for progressively unrolling the blister strip 20 and for causing it to advance. The base-layer portion 22 including the empty cavities is advantageously adapted to be rolled up in another location of said body 10, in particular a first receiver portion, and the unstuck closure-layer portion 23 is advantageously adapted to be rolled up in yet another location of said body 10, in particular a second receiver portion.

The first reception portion advantageously comprises a first rotary receiver element 40, around which the base-layer portion 22 including the empty cavities is rolled up. Advantageously, the first rotary receiver element 40 is urged to turn by a spring (not shown), in particular a spiral spring that is arranged inside said first rotary element. This guarantees proper rolling up on each actuation.

The second receiver portion advantageously comprises a second rotary receiver element 50, around which the unstuck closure-layer portion 23 rolls up. Advantageously, the second rotary receiver element 50 is also urged to turn, in particular being correlated in rotation with the displacement means 30 of the blister strip 20. This guarantees proper rolling up on each actuation.

The displacement means 30 are adapted to cause the blister strip 20 to advance before and/or during each actuation of the device. The displacement means preferably comprise an indexer wheel 30 that receives and guides the blisters. Turning the indexer wheel 30 causes the blister strip 20 to advance. Advantageously, the indexer wheel 30 is turned when the device is primed, e.g. by opening the cap.

In an advantageous aspect, the inhaler includes a dispersion chamber 70 for receiving the dose of powder after a respective reservoir has been opened. The dispersion chamber 70 includes an inlet 710 that is connected directly to the open reservoir via a powder delivery channel 60, and an outlet 720 that is connected directly to the dispenser orifice 15 via a powder dispenser channel 80. The dispersion chamber 70 preferably contains at least one ball 71, advantageously a plurality of balls, e.g. three balls. The balls 71 may move in said dispersion chamber 70 along a ball path 75 that is substantially circular and that extends in a plane that is approximately transverse to the orientation of the delivery and dispenser channels 60 and 80. Thus, said delivery and dispenser channels extend in the same direction that is substantially perpendicular to said transverse plane of said ball path. The dispersion chamber 70 includes at least one, preferably two air inlets 72. The air inlets 72 are preferably formed by air channels 90 that open out into the dispersion chamber 70 in substantially tangential manner. Thus, the tangential air inlets are arranged in said transverse plane of said ball path. They are thus substantially perpendicular to said delivery and dispenser channels. In this way, the flows of air entering via the air channels 90 swirl in the dispersion chamber 70 and cause the balls 71 to turn on the ball path 75. The flows of air that are created by the user inhaling are mixed with the flow of air and of powder that comes from the open reservoir through the delivery channel 60, and that penetrates into the dispersion chamber 70 via the inlet 710. The flow of air and of powder is thus also caused to turn in the dispersion chamber 70, and the balls 71 break up the powder before said powder is driven from the dispersion chamber 70 via said outlet 720 from where it is transferred to the dispenser orifice 15 via said dispenser channel 80. In the position shown in FIG. 1, the delivery and dispenser channels 60 and 80 extend in the same direction, which is the vertical direction in FIG. 1, and the ball path 75 and the air channels 90 extend in a plane that is substantially horizontal in the position in FIG. 1.

In the embodiment in FIGS. 2 and 3, the delivery channel 60 is substantially coaxial with the dispenser channel 80, the two channels opening out into the dispersion chamber 70 substantially at its center. In order to avoid the flow of air and of powder that comes from the open reservoir flowing directly into the dispenser channel 80, said inlet 710 of the dispersion chamber 70 includes a deflector 100 that deflects said flow of air and of powder towards said ball path 75 of the dispersion chamber 70. By way of example, the deflector 100 may be formed by a curved wall portion in the shape of a dish having an outer tip that faces the incoming flow of air and of powder. A more angular shape may also be envisaged as a variant to the rounded shape that is shown, and other more complex deflector shapes are also possible, the essential being to deflect the flow of air and of powder towards the dispersion chamber 70, and thus avoid it flowing in a straight line directly into the dispenser channel 80.

In another advantageous embodiment, shown in FIGS. 5 and 7, the dispenser channel 80 extends in similar manner to the first embodiment, i.e. substantially vertically from the center of the dispersion chamber 70 (in the position in FIG. 1). However, the delivery channel 60 is offset relative to the dispenser channel 80, unlike the first embodiment in which they are in alignment. Thus, as can be seen in particular in FIG. 5, the delivery channel 60 opens out directly into the ball path 75 of the dispersion chamber 70. The inlet 710 thus includes a profile 715, such as a mesh, so as to prevent the balls 71 from falling into said delivery channel 60. In this variant, a deflector is no longer necessary, since the flow of air and of powder opens out directly into the ball path 75 in which it is caused to turn by the flow of air and by the balls that turn. Nevertheless, it is still possible to envisage using a deflector to direct, at least in part, the flow of air and of powder towards the outer portion of the ball path, if necessary.

When the device is actuated, the indexer wheel 30 is turned, and this brings the next blister 21 to face the inlet 61 of the delivery channel 60. Simultaneously, the closure-layer 23 is removed from the blister 21, being peeled off by turning the second rotary receiver element 50 that is advantageously correlated in rotation with the indexer wheel 30. Advantageously, the closure layer extends around a stationary peeling corner 18 that, in association with the simultaneous turning of the indexer wheel 30 and of the second rotary receiver element 50, causes the closure layer 23 to be unstuck from or peeled off the base layer 22 at said peeling corner 18. The base layer 22 downstream from the indexer wheel 30 rolls up around the first rotary receiver element 40. Thus, an open blister 21 is situated facing the delivery channel 60. As can be seen in FIGS. 2, 3, 5, and 7 in particular, the blisters 21 extend transversally relative to the blister strip 20. In this way, when the open blister faces the inlet 61 of the delivery channel 60, the inlet 61 extends over approximately half of the length of the blister, and the other half of the length is situated facing the outlet 96 of an air delivery channel 95. Thus, when the user inhales, a first flow of air is created through said air delivery channel 95.

This first flow of air passes through the open blister 21, as shown by arrow A1 in FIGS. 2 and 5, and thus entrains the powder in the form of a flow of air and of powder into the delivery channel 60, as shown by arrows B. Simultaneously, the inhalation creates secondary flows of air that penetrate tangentially into the dispersion chamber 70 through the tangential air channels 90, as shown in FIGS. 4 and 8 by arrows A2. The secondary flows of air cause the balls 71 to turn in the dispersion chamber 70. When the flow of air and of powder encounters the secondary flows of air and the balls moving in the dispersion chamber 70, swirls are created, shown by arrows C in FIGS. 2 and 5. This fluidifies and breaks up the powder that finally escapes towards the dispenser orifice 15, as shown by arrows D in FIGS. 2 and 5.

Naturally, the dimensions and orientations of the various channels may be optimized depending on the features of the inhaler, so as to maximize the performance thereof.

Various modifications are possible for the skilled person without departing from the scope of the present invention as defined in the accompanying claims.

1. A powder inhaler device comprising: a body that is provided with a dispenser orifice; a plurality of predosed reservoirs each containing a dose of powder for dispensing; and reservoir-opening means for opening a reservoir on each actuation; the device further comprising a dispersion chamber including an inlet connected during inhalation to an open reservoir and receiving the flow of air and of powder from said open reservoir via a delivery channel, and an outlet connected to said dispenser orifice via a dispenser channel, said dispersion chamber including at least one ball that is movable along a ball path in said dispersion chamber, said dispersion chamber including at least one air inlet that is approximately tangential to said ball path, said device being characterized in that said delivery and dispenser channels extend in a same direction that is substantially perpendicular to said ball path and to said at least one tangential air inlet.

2. A device according to claim 1, wherein said delivery channel is substantially coaxial with said dispenser channel, said inlet and outlet of the dispersion chamber being arranged substantially at the center of said dispersion chamber.

3. A device according to claim 2, wherein said inlet of the dispersion chamber includes a deflector so as to deflect the flow of air and of powder towards the ball path of said dispersion chamber.

4. A device according to claim 3, wherein said deflector is dish shaped with the outer tip of said dish facing the incoming flow of air and of powder.

5. A device according to claim 1, wherein said delivery channel is offset relative to said dispenser channel, said inlet of the dispersion chamber being arranged at the ball path, and said outlet of the dispersion chamber being substantially at the center of said dispersion chamber.

6. A device according to claim 5, wherein said inlet of the dispersion chamber includes a mesh.

7. A device according to claim 1, wherein each air inlet of the dispersion chamber is formed by a tangential air channel.

8. A device according to claim 1, wherein said plurality of reservoirs is formed on an elongate flexible blister strip comprising a base layer containing the cavities of the reservoirs, and a closure layer overlying said cavities.

9. A device according to claim 8, wherein the closure layer is peelable off the base layer, the portion of base layer containing the empty blisters rolling up around a first rotary receiver element, and the portion of closure layer peeled off said base layer rolling up around a second rotary receiver element.

10. A device according to claim 9, wherein said reservoir opening means include means for unsticking said closure layer from said base layer, such as a peeling edge around which said closure layer extends, the device including displacement means, such as an indexer wheel, so as to cause the blister strip to advance before and/or during each actuation, the downstream end of said closure layer being fastened to a second rotary receiver element that rotates in correlation with said indexer wheel.

11. A device according to claim 1, wherein said dispersion chamber contains a plurality of balls.

12. A device according to claim 1, wherein, after opening, said open reservoir is situated facing both said delivery channel and an air delivery channel, such that during inhalation, a flow of air penetrates into said blister through said air delivery channel and entrains said powder in a flow of air and of powder via said delivery channel towards said dispersion chamber.
